(19) United States
(12) Patent Application Publication
(10) Pub. No.: US 2020/0080979 A1
(43) Pub. Date: Mar. 12, 2020

(54) METHOD AND DEVICES FOR THE SPECTROPHOTOMETRIC DETERMINATION OF RESIDUAL PHASE TRANSFER CATALYST IN A PET RADIOPHARMACEUTICAL DOSE

(71) Applicant: University of Tennessee Research Foundation, Knoxville, TN (US)

(72) Inventors: David Blevins, Powell, TN (US); Dustin Ryan Osborne, Knoxville, TN (US)

(21) Appl. No.: 16/563,474

(22) Filed: Sep. 6, 2019

Related U.S. Application Data
(60) Provisional application No. 62/727,837, filed on Sep. 6, 2018.

(51) Int. Cl. G01N 33/15; G01N 1/40; B01J 31/02; G01N 21/31; G01N 27/06

(52) U.S. Cl. CPC G01N 33/15; G01N 1/4055; B01J 31/0204; G01N 2033/0093; G01N 21/31; G01N 27/06; B01J 31/0239

(57) ABSTRACT

Highly quantitative methods for determining the concentration of residual phase transfer catalysts (PTCs) in radiotracer or radiopharmaceutical doses are described. The methods comprise mixing aliquots of the doses that can contain residual PTCs with a sodium and/or potassium salt; extracting a residual PTC/salt complex into an organic phase; and detecting the amount of PTC/salt complex in the organic phase. The detecting can involve visual colorimetry or measuring the absorbance or transmittance of the organic phase when the sodium and/or potassium salt comprises a chromophoric ion, or measuring the resistance of the organic phase. Also described are devices for use in performing the methods.

### RELATED APPLICATIONS

This application claims the benefit of U.S. Provisional Patent Application Ser. No. 62/727,837, filed Sep. 6, 2018; the disclosure of which is incorporated herein by reference in its entirety.

### TECHNICAL FIELD

The presently disclosed subject matter relates to methods and devices for detecting the presence or concentration of phase transfer catalysts (PTCs) in samples. In some embodiments, the methods and devices can be used to detect the concentration of residual PTC in a radiotracer or a radiopharmaceutical.

### ABBREVIATIONS

%=percentage
° C.=degrees Celsius
μL=microliter
¹⁸F=fluorine-18
DCM=dichloromethane
kg=kilogram
KMnO₄=potassium permanganate
LD₅₀=lethal dose, 50%
LED=light-emitting diode
M=molar
mg=milligram
min=minute
mL=milliliter
mm=millimeter
Mohm=megaohm
nm=nanometer
PET=positron emission tomography
ppm=parts-per-million
QC=quality control
s=seconds
TBA=tetrabutylammonium cation
TBAHC=tetrabutylammonium hydrogen carbonate
TLC=thin layer chromatography
USP=United States Pharmacopeia
UV-Vis=ultraviolet-visible

### BACKGROUND

In the manufacture of fluorine-18 (¹⁸F)-labeled radiotracers and radiopharmaceuticals using medical cyclotron produced ¹⁸F, a phase transfer catalyst (PTC), such as the cryptand KRYPTOFIX™-222, (Merck KGAA, Darmstadt, Germany), i.e., 4,7,13,16,21,24-dexaoxa-1,10-diazabicyclo[8.8.8]hexacosane (also known as K-222 or cryptand 222); or tetrabutylammonium hydrogen carbonate (TBAHC) can be used to complex the cation of ¹⁸F-potassium fluoride so that it can be dissolved in an organic reaction solvent. During the radio-synthesis, the radiotracer or radiopharmaceutical product is purified and formulated into the final dose.

Although the purification process is designed to remove impurities in the final dose, residual PTC is often present. As a quality control (QC) measure, a thin layer chromatography (TLC) spot is compared to a known PTC standard solution of 50 parts-per-million (ppm). In the spot test, the standard and the radiotracer or radiopharmaceutical preparation are both spotted adjacently onto a silica or alumina TLC plate, and then a stain (e.g., iodine or iodoplatinate) is applied to visualize the spots. For the radiotracer/radiopharmaceutical dose to pass this QC test, its intensity should be equal to or less than the standard. This subjective, semi-quantitative test is the current standard for residual PTC testing for K-222.

Although the crown ether 18-crown-6 can be superior to K-222 as a PTC for radiolabeling reactions by providing higher radiochemical yields, there is no currently accepted QC test for this more toxic compound in radiotracers and radiopharmaceuticals. Because of these factors, it is not currently utilized in clinical radiochemistry.

Accordingly, there is an ongoing need for additional methods and devices for determining residual PTC in radiotracer/radiopharmaceutical doses. In particular, there is an ongoing need for methods that can be applied to a broad range of PTCs, that are inexpensive and easy to use, and that provide a more accurate, non-subjective measure of residual PTC concentration, particularly at lower concentrations (e.g., less than 50 ppm).

### SUMMARY

In some embodiments, the presently disclosed subject matter provides a method of detecting the presence or concentration of a phase transfer catalyst (PTC) in a sample, the method comprising: (a) mixing a sample containing or suspected of containing a PTC with an aqueous solution comprising a potassium and/or sodium salt to provide an aqueous mixture; (b) adding an organic solvent to the aqueous mixture to provide a biphasic mixture comprising an aqueous phase and an organic phase; (c) mixing the biphasic mixture for a period of time; (d) separating the organic phase from the aqueous phase; and (e) analyzing the organic phase, thereby determining the presence or concentration of the PTC. In some embodiments, the sample containing or suspected of containing a PTC comprises a radiopharmaceutical.

In some embodiments, the radiopharmaceutical comprises fluorine-18 (¹⁸F). In some embodiments, the radiopharmaceutical is selected from the group comprising [¹⁸F]2-fluoro-2-deoxy-D-glucose (FDG) [¹⁸F]sodium fluoride; [¹⁸F]3′-deoxy-3′-fluorothymidine (FLT), [¹⁸F]fluoromisonidazole, [¹⁸F]florbetaben, [¹⁸F]florbetapir, [¹⁸F]fluoro-ethyl-tyrosine (FET), [¹⁸F]flutemetamol, [¹⁸F]-fluorocholine (FCH), [¹⁸F]fluoroethylcholine (FECH), [¹⁸F]fallypride, and [¹⁸F]6-fluor-L-2,3-dihydroxyphenylalanine (FDOPA).

In some embodiments, the PTC is selected from a quaternary ammonium salt, a cryptand, and a crown ether. In some embodiments, the PTC is selected from the group comprising K-222, tetrabutylammonium hydrogen carbonate, and 18-crown-6. In some embodiments, the organic solvent is dichloromethane. In some embodiments, step (c) comprises vortexing the biphasic mixture for about 30 seconds.

In some embodiments, the potassium and/or sodium salt is the potassium or sodium salt of a chromophoric anion. In some embodiments, the potassium and/or sodium salt is selected from the group comprising potassium permanganate and sodium resazurin.

In some embodiments, step (e) comprises measuring the light absorbance of the organic phase at one or more wavelengths of interest and comparing the light absorbance of the organic phase to the light absorbance of one or more standard solutions, wherein each of the one or more standard solutions comprises a known concentration of a complex of the PTC and the salt dissolved in the organic solvent. In some embodiments, the potassium and/or sodium salt is selected from the group comprising potassium permanganate and sodium resazurin, and the one or more wavelengths of interest is 532 nanometers (nm). In some embodiments, the aqueous solution comprising the potassium and/or sodium salt comprises about 0.1 and about 0.5 molar (M) potassium permanganate, optionally wherein the aqueous solution comprising the potassium and/or sodium salt comprises about 0.2 M potassium permanganate.

In some embodiments, the measuring is performed using a spectrophotometric device comprising a green laser and a light detector. In some embodiments, the device further comprises one or more of a reservoir for the organic phase, a microprocessor, a solid body for holding a sample reservoir in the path of a beam of light from the green laser, and a display for displaying one or more absorbance measurement values.

In some embodiments, step (e) comprises measuring the electrical conductivity of the organic phase and comparing the electrical conductivity of the organic phase to the electrical conductivity of one or more standard solutions, wherein each of the one or more standard solutions comprises a known concentration of a complex of the PTC and the salt dissolved in the organic solvent. In some embodiments, the potassium and/or sodium salt comprises a mixture of sodium resazurin and potassium carbonate. In some embodiments, the aqueous solution comprising the potassium or sodium salt comprises between about 0.02 and about 0.06 molar (M) sodium resazurin and between about 0.02 and about 0.06 M potassium carbonate. In some embodiments, the aqueous solution comprises equimolar concentrations of the sodium resazurin and the potassium carbonate, optionally wherein both the sodium resazurin and the potassium carbonate have a concentration of about 0.05 M. In some embodiments, the measuring is performed with a photodiode resistor or a multimeter.

In some embodiments, the sample has a volume of between about 50 microliters (μL) and about 100 μL, and the aqueous solution comprising a potassium and/or sodium salt has a volume of about 50 μL. In some embodiments, adding an organic solvent comprises adding about 1 milliliter (mL) of the organic solvent.

In some embodiments, the potassium and/or sodium salt is the potassium or sodium salt of a chromophoric anion and step (e) comprises visually comparing the color of the organic phase to the color of one or more standard solutions, wherein each of the one or more standard solutions comprises a known concentration of a complex of the PTC and the salt dissolved in the organic solvent.

In some embodiments, the presently disclosed subject matter provides a method of conducting a quality control test on a radiopharmaceutical, wherein the method comprises: (a) mixing an aliquot of a radiopharmaceutical solution with an aqueous solution comprising a potassium and/or sodium salt to provide an aqueous mixture; (b) adding an organic solvent to the aqueous mixture to provide a biphasic mixture comprising an aqueous phase and an organic phase; (c) mixing the biphasic mixture for a period of time; (d) separating the organic phase from the aqueous phase; and (e) analyzing the organic phase, thereby determining the concentration of a residual phase transfer catalyst (PTC) in the radiopharmaceutical. In some embodiments, the analyzing comprises (i) measuring an optical absorbance of the organic phase or an electrical conductivity of the organic phase, and (ii) comparing the optical absorbance or electrical conductivity to an optical absorbance or electrical conductivity of one or more standard solutions, wherein each of the one or more standard solutions comprises a known concentration of the PTC complexed to the potassium and/or sodium salt.

In some embodiments, the residual PTC is selected from K-222, tetrabutylammonium hydrogen carbonate, and 18-crown-6. In some embodiments, the radiopharmaceutical is selected from the group comprising [¹⁸F]2-fluoro-2-deoxy-D-glucose (FDG) [¹⁸F]sodium fluoride; p deoxy-3′-fluorothymidine (FLT), [¹⁸F]fluoromisonidazole, [¹⁸F]florbetaben, [¹⁸F]florbetapir, [¹⁸F]fluoro-ethyl-tyrosine (FET), [¹⁸F]flutemetamol, [¹⁸F]-fluorocholine (FCH), [¹⁸F]fluoroethylcholine (FECH), [¹⁸F]fallypride, and [¹⁸F]6-fluor-L-2,3-dihydroxyphenylalanine (FDOPA).

Accordingly, it is an object of the presently disclosed subject matter to provide a method for detecting the presence or concentration of a PTC in a sample or of conducting a quality control test on a radiopharmaceutical.

An object of the presently disclosed subject matter having been stated hereinabove, and which is achieved in whole or in part by the presently disclosed subject matter, other objects will become evident as the description proceeds herein below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a schematic drawing showing a top view of an exemplary device for spectrophotometric determination of residual phase transfer catalyst (PTC).

FIG. 1B is a schematic drawing showing a side view of an exemplary device for spectrophotometric determination of residual phase transfer catalyst (PTC).

FIG. 2 is a schematic drawing showing a side view of an exemplary device for the electrochemical measurement of residual phase transfer catalyst (PTC).

FIG. 3A is a graph showing the absorbance (at 332 nanometers (nm)) of organic solutions comprising complexes of K-222 (Kryptofix-222) and potassium permanganate extracted from mixtures of an aqueous solution of 0.2 molar (M) potassium permanganate and aqueous solutions of K-222 with concentrations up to 250 parts-per-million (ppm).

FIG. 3B is a graph showing the resistance of organic solutions comprising complexes of 18-crown-6 and potassium permanganate extracted from mixtures of an aqueous solution of 0.2 molar (M) potassium permanganate and aqueous solutions of 18-crown-6 with concentrations of up to 250 parts-per-million (ppm).

FIG. 4A is a composite photograph showing an organic (dichloromethane) phase comprising extracted resazurin dye from: (top) aqueous mixtures of K-222 and sodium resazurin solutions where the K-222 solutions contained, from left to right, 0, 12, 25, 50, 75, 100, and 150 parts-per-million (ppm) K-222; (middle) aqueous mixtures of tetrabutylammonium hydrogen carbonate (TBAHC) and sodium resazurin solutions where the TBAHC solutions contained, from left to right, 0 12, 25, 50, 75, 100, and 150 ppm K-222; and (bottom) aqueous mixtures of 18-crown-6 and sodium resazurin solutions where the 18-crown-6 solutions contained, from left to right, 0, 12, 25, 50, 75, 100, and 150 ppm 18-crown-6.

FIG. 4B is a photograph showing examples of thin layer chromatography spot testing of K-222 standards stained with iodoplatinate stain. From left to right, the K-222 standard contained 0, 40, 50, and 60 parts-per-million (ppm) K-222.

FIG. 5A is a graph showing the transmittance (reported as percentage (%) transmittance) of sodium resazurin complexes of K-222 (diamonds), 18-crown-6 (circles), and tetrabutylammonium hydrogen carbonate (squares with “X”s) at 532 nanometers (nm) in dichloromethane extracted from mixtures prepared from phase transfer catalyst solutions containing 0, 6, 12, 25, 50, 75, 100, and 150 parts-per-million (ppm), as indicated on the x-axis.

FIG. 5B is a graph showing the effect of sodium resazurin concentration on the extraction of sodium resazurin/phase transfer catalysts from aqueous mixtures. The data provides absorbance values (in arbitrary units) versus wavelength (in nanometers) of dicloromethane extracts from mixtures of a 100 parts-per-million (ppm) K-222 solution and solutions containing equimolar amounts of sodium resazurin and potassium carbonate, where the concentration of the sodium resazurin and potassium carbonate was 0.02 (unfilled dotted and dashed line), 0.03 (filled dotted and dashed line), 0.04 (filled dashed line), 0.05 (filled dotted line), or 0.06 (filled solid line) moles per liter (M).

FIG. 5C is a graph showing the effect of potassium carbonate concentration on the extraction of sodium resazurin/phase transfer catalysts from aqueous mixtures. The data provides absorbance values (in arbitrary units) versus wavelength (in nanometers) of dicloromethane extracts from mixtures of a 100 parts-per-million (ppm) K-222 solution and solutions containing 0.05 moles per liter (M) sodium resazurin and potassium carbonate, where the concentration of the potassium carbonate was 0.01 (filled dashed line), 0.02 (filled dotted and dashed line), 0.03 (unfilled dashed line), 0.04 (unfilled dotted and dashed line), or 0.05 (filled dotted line) M.

FIG. 5D is a graph showing the transmittance (measured as a percentage) of extracts obtained from mixtures of a solution of sodium resazurin and potassium carbonate and a solution of 50 parts-per-million (ppm) K-222 containing either 0 ppm acetonitrile (ACN, filled solid line) or 400 ppm ACN (dashed line).

FIG. 5E is a graph showing the transmittance (measured as a percentage) of extracts obtained from mixtures of a solution of sodium resazurin and potassium carbonate and a solution of 50 parts-per-million (ppm) K-222 containing either 1250 ppm ethanol (filled solid line) or 5000 ppm ethanol (dashed line).

FIG. 6A is a graph showing the transmittance (at 532 nanometers) of dichloromethane solutions comprising complexes of sodium resazurin and tetrabutylammonium hydrogen carbonate extracted from mixtures of an aqueous solution of 0.04 molar (M) sodium resazurin and 0.04 M potassium carbonate and aqueous solutions of tetrabutylammonium hydrogen carbonate with concentrations between 10 and 150 parts-per-million (ppm).

FIG. 6B is a graph showing the transmittance (at 532 nanometers) of dichloromethane solutions comprising complexes of sodium resazurin and K-222 (Kryptofix-222) extracted from mixtures of an aqueous solution of 0.04 molar (M) sodium resazurin and 0.04 M potassium carbonate and aqueous solutions of K-222 with concentrations between 10 and 150 parts-per-million (ppm).

FIG. 6C is a graph showing the transmittance (at 532 nanometers) of dichloromethane solutions comprising complexes of sodium resazurin and 18-crown-6 extracted from mixtures of an aqueous solution of 0.04 molar (M) sodium resazurin and 0.04 M potassium carbonate and aqueous solutions of 18-crown-6 with concentrations of between 10 and 150 parts-per-million.

FIG. 7A is a graph showing the resistance (in megaohms (Mohm)) of dichloromethane solutions comprising complexes of sodium resazurin and 18-crown-6 extracted from mixtures of an aqueous solution of 0.04 molar (M) sodium resazurin and 0.04 M potassium carbonate and aqueous solutions of 18-crown-6 with concentrations between 10 and 100 parts-per-million (ppm).

FIG. 7B is a graph showing the resistance (in megaohms (Mohm)) of dichloromethane solutions comprising complexes of sodium resazurin and K-222 (Kryptofix-222) extracted from mixtures of an aqueous solution of 0.04 molar (M) sodium resazurin and 0.04 M potassium carbonate and aqueous solutions of K-222 with concentrations between 10 and 100 parts-per-million (ppm).

FIG. 7C is a graph showing the resistance (in megaohms (Mohm)) of dichloromethane solutions comprising complexes of tetrabutylammonium hydrogen carbonate and sodium resazurin extracted from mixtures of an aqueous solution of 0.04 molar (M) sodium resazurin and 0.04 M potassium carbonate and aqueous solutions of tetrabutylammonium hydrogen carbonate with concentrations between 10 and 100 parts-per-million (ppm).

FIG. 8 is a graph of the averaged transmittance and resistance (measured in megaohms (Mohms) data for complexes of all the phase transfer catalysts (PTC) tested extracted from aqueous mixtures prepared from a solution of sodium resazurin and potassium carbonate as a function of the concentration (in parts-per-million (ppm)) of the PTC in the sample mixed with the sodium resazurin/potassium carbonate solution. Resistance data when the PTC was 18-crown-6 is shown by “x”s, resistance data when the PTC was tetrabutylammonium hydrogen carbonate is shown by “ [symbol] ”s, resistance data when the PTC was K-222 is shown by circles, transmittance data when the PTC is K-222 is shown by squares, transmittance data when the PTC is tetrabutylammonium hydrogen carbonate is shown by triangles, and transmittance data when the PTC is 18-crown-6 is shown by diamonds.

### DETAILED DESCRIPTION

The presently disclosed subject matter will now be described more fully. The presently disclosed subject matter can, however, be embodied in different forms and should not be construed as limited to the embodiments set forth herein below and in the accompanying Examples. Rather, these embodiments are provided so that this disclosure will be thorough and complete, and will fully convey the scope of the embodiments to those skilled in the art.

All references listed herein, including but not limited to all patents, patent applications and publications thereof, and scientific journal articles, are incorporated herein by reference in their entireties to the extent that they supplement, explain, provide a background for, or teach methodology, techniques, and/or compositions employed herein.

### I. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs.

Following long-standing patent law convention, the terms “a”, “an”, and “the” refer to “one or more” when used in this application, including the claims.

The term “and/or” when used in describing two or more items or conditions, refers to situations where all named items or conditions are present or applicable, or to situations wherein only one (or less than all) of the items or conditions is present or applicable.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” can mean at least a second or more.

The term “comprising”, which is synonymous with “including,” “containing,” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

Unless otherwise indicated, all numbers expressing quantities of time, absorbance, transmittance, resistance, wavelength, concentration, volume and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about”, when referring to a value is meant to encompass variations of in one example ±20% or ±10%, in another example ±5%, in another example ±1%, and in still another example ±0.1% from the specified amount, as such variations are appropriate to perform the disclosed methods.

Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4).

The term “complex” as used herein refers to an entity formed via non-covalent interactions between at least two chemical species, such as between an organic molecule and another organic molecule, salt, or an ion (e.g., a cation). In some embodiments, the complex can comprise one or more coordinate bond between a cation and an organic molecule ligand comprising an electron pair donor, ligand or chelating group. Thus, the organic molecule (which can also be referred to as a ligand or chelating group) generally comprises one or more electron pair donors, molecules or molecular ions having atoms (e.g., oxygen or nitrogen atoms) with unshared electron pairs available for donation to a cation, such as a sodium or potassium ion.

The term “coordinate bond” refers to an interaction between an electron pair donor and a coordination site on a cation resulting in an attractive force between the electron pair donor and the cation. The use of this term is not intended to be limiting, in so much as certain coordinate bonds also can be classified as have more or less covalent character (if not entirely covalent character) depending on the characteristics of the metal ion and the electron pair donor.

The term “phase transfer catalyst” (or PTC) as used herein refers to a chemical compound or species that facilitates the migration of a chemical reagent from one phase (e.g., an aqueous phase) to another phase (e.g., an organic phase). Suitable PTCs include, but are not limited to quaternary ammonium salts, crown ethers, and cryptands.

The term “quaternary ammonium salt” as used herein refers to a compound with the formula NR₄⁺X⁻ wherein each R is independently an alkyl or aryl group and X is an anion that can dissociate from the cation in an aqueous environment, as well as to the cation thereof (i.e., the cation with the formula NR₄⁺). The cation can also be referred to herein as a quaternary ammonium species. In some embodiments X⁻ is a halogen anion (e.g., chloride, bromide, or iodide), a bicarbonate (or hydrogen carbonate, i.e., HCO₃), or a hydroxyl anion. Exemplary quaternary ammonium salts suitable for use as PTCs include, but are not limited to, tetrabutylammonium hydrogen carbonate, tri-n-butyl-methylammonium chloride, phenyltrimethylammonium bromide, tetra-n-butylammonium bromide, tetraethylammonium chloride, triethylbenzylammonium chloride, ethyltrimethylammonium iodide, trimethyloctodecylammonium chloride, trimethyldodecylammonium chloride, tetra-n-propylammonium chloride, methyltriocylammonium chloride, and the cations thereof.

The term “crown ether” as used herein refers to a cyclic polyether. Exemplary crown ethers include cyclic oligomers of ethylene oxide. In some embodiments, two carbon atoms of an alkylene moiety of a cyclic polyether can be replace by two carbon atoms from an aryl moiety, such as phenyl or naphthyl, which can be substituted or unsubstituted at the carbons not forming part of the backbone of the cyclic polyether. The oxygen atoms of the crown ethers can coordinatively bind to cations, thereby forming complexes with the cations or their salts. The crown ethers can act as multidentate ligands for cations. An exemplary crown ether is 18-crown-6, where 18 is the total number of atoms in the backbone of the cyclic polyether and 6 is the number of oxygen atoms in the backbone of the cyclic polyether. Additional exemplary crown ethers include, but are not limited to, 15-crown-5, benzo-15-crown-5, 12-crown-4, and dibenzo-18-crown-6.

The term “cryptand” as used herein refers to a bicyclic or polycyclic multidentate ligand for a cation or a salt thereof. An exemplary cryptand is [2,2,2]cryptand (i.e., K-222), wherein each 2 indicates a number of oxygen atoms in a polyether bridge between two nitrogen atoms. In some embodiments, the polyether bridge can include one or more arylene moiety. In some embodiments, the oxygen atoms of the polyether bridges can be replaced by nitrogen atoms.

The terms “radionuclide”, “radioactive isotope” and “radioisotope” refer to an unstable atom that has excess nuclear energy. Radionuclides lose the excess energy via a radioactive decay process (e.g., positron emission or beta decay), forming a stable nuclide or another radionuclide that can then decay to form a stable nuclide.

The term “radiotracer” refers to an imaging agent, e.g., used in medicine or in veterinary practice, that comprises a radionuclide.

The term “radiopharmaceutical” refers to a pharmaceutical compound (i.e., a compound that provides a beneficial therapeutic effect in treating a medical or veterinary disease or condition) that comprises a radionuclide.

The term “chromophoric” as used herein refers to an ionic species, organic compound, or a group within an organic compound or ionic species that absorbs light (e.g., visible light) at a particular wavelength (e.g., a particular wavelength between 400 and 700 nm) and, thus, makes the species or compound appear colored. For example, chromophoric anions based on organic compounds typically include a conjugated system of alternating double and single bonds that can provide resonance stabilization and a group that provides a negative charge (e.g., a carboxylate or sulfonate).

### II. General Considerations

Crown ethers and cryptands are soluble in both aqueous and lipophilic organic solvents and are used as PTCs in chemistry. Nucleophilic fluorinations frequently employ 18-crown-6 ether as the PTC for potassium fluoride, and nucleophilic radio-fluorinations typically utilize the cryptand KRYPTOFIX™ (Merck KGAA, Darmstadt, Germany; also referred to herein as K-222) as the PTC for the production of ¹⁸F-labeled radiotracers and radiopharmaceuticals. K-222 is typically preferred in radiochemistry because instead of a 2-dimensional cation complexation, it forms a 3-dimensional complex that is 10⁴ times more stable than the corresponding 18-crown-6/potassium cation complex. As a result of the more solvated ion pair, the fluoride reactivity is increased, which is important in no-carrier-added radiofluorinations where the [¹⁸F]fluoride is present in low nanomole quantities [1-6]. Quaternary ammonium salts, such as tetrabutylammonium hydrogen carbonate (TBAHC) and tetraethylammonium hydrogen carbonate are also PTCs used in nucleophilic fluorination chemistry [7].

During radiopharmaceutical production of ¹⁸F-labeled radiotracers and radiopharmaceuticals for clinical use, quality control (QC) spot tests are used to verify that the K-222 or tetrabutylammonium cation (TBA) is at or below the United States Pharmacopeia (USP) established limit of 50 ppm. The 50 ppm limit for K-222 and TBA has been set because these PTCs are toxic compounds. 18-Crown-6 currently has no USP limit or an accepted test, and is not employed in the production of clinical radiotracers or radiopharmaceuticals because it has higher toxicity than K-222. 18-Crown-6 has been shown to be more toxic than K-222 in animals (oral LD₅₀ 525 mg/kg vs up to 2000 mg/kg in rats); and in rabbits, doses as low as 6 mg/kg lead to neurological symptoms. Based on this data, it is presumable that an acceptable level would likely be set at around 25 ppm [8]. However, in development on the radiosynthesis of [¹⁸F]fluorocholine, the use of 18-crown-6 as the PTC instead of K-222 has been found to provide a much higher radiochemical yield of the radiotracer. Thus, a PTC QC method that allows more precise quantification and verification of low levels of 18-crown-6 in radiopharmaceuticals would likely facilitate its adoption and utilization when advantageous.

The current QC spot test consists of spotting a K-222 or TBA 50 ppm standard onto a silica thin layer chromatography plate, and then adjacently spotting the radiotracer or radiopharmaceutical formulation. Once the spots have dried, they are visualized with iodine vapor or, in the case of K-222, an iodoplatinate solution can be used instead. If the intensity of the radiotracer spot is equal to or less dark than the standard spot, the dose passes. This qualitative test is somewhat subjective, but there is currently no widely used, highly quantitative method utilized for routine PTC QC testing of radiotracers and radiopharmaceuticals [9]. Thus, it can be difficult, for example, to optimize the synthesis of radiotracers/radiopharmaceuticals to avoid higher residual PTC concentrations. In addition, K-222 spot test “false positives” are possible with radiotracers/radiopharmaceuticals containing tertiary amine functions, and “false negatives” may occur when stabilizers are added to the radiotracer/radiopharmaceutical preparation. [10].

In an effort to meet the need for an alternative analysis method for PTC testing, two analytical instrumental methods (i.e., gas chromatography and high-performance liquid chromatography) have been evaluated for measuring residual K-222 levels. A main drawback of these methodologies is the expensive equipment required [11,12]. Recently, a microfluidic “spectroscopy chip” was developed for use with a microfluidic radiosynthesis system that uses iodoplatinate as a test reagent. It allows spectrophotometric measurement of K-222 in radiopharmaceuticals with a limit of detection of 28 ppm [13]. Additionally, another spectrophotometric method has been investigated that involves measuring the absorbance of a K-222/7, 7, 8, 8-Tetracyanoquinodimethane charge transfer complex. It was found to have a working range of 0-30 ppm [14]. Currently, there is one FDA approved automated radiopharmaceutical quality-control testing platform (TRACER-QC™ by Trace-Ability, Inc., Culver City, Calif., United States of America). This machine has the capability to carry out all QC testing (including K-222 QC using an iodoplatinate-based optical limit test) for [¹⁸F]2-fluorodeoxyglucose ([¹⁸F]FDG) production, but is not in wide use because the cost of the device is prohibitively expensive for many academic and commercial radiopharmacies. This QC testing platform also has the limitation that it is not able to analyze for TBA when applied to radiotracers/radiopharmaceuticals that use this PTC.

In some embodiments, the presently disclosed subject matter provides a method for quantitively testing for the presence or concentration of a PTC in a sample suspected of, or known to contain, a PTC. In some embodiments, the sample contains PTC at a concentration of less than 150 ppm, less than 100 ppm, or less than 50 ppm. In some embodiments, the sample is a radiotracer or radiopharmaceutical dose formulation and the method can determine the concentration of residual PTC in the sample (i.e., the amount of PTC remaining after the synthesis of the PTC). Macrocyclic PTCs allow the dissolution of inorganic salts, such as potassium [¹⁸F]fluoride in organic solvents by enclosing the potassium ion in the interior of their cage-like structure, which disperses the charge of the potassium ion over a greater area, facilitating solubility in a relatively nonpolar solvent medium. In the case of organic salt PTCs, such as the quaternary ammonium salt tetrabutylammonium hydrogen carbonate, the hydrogen carbonate anion forms hydrogen bonds with the negatively charged fluoride and this negative complex is surrounded by positive tetrabutylammonium cations, allowing dispersion of the charge and dissolution. The presently disclosed methods are based on the ability of PTCs to solubilize organic salts in relatively nonpolar organic solvents as a means of quantification. Quantification methods can include visual colorimetry, spectrophotometric analysis, or measurement of electrical conductance through the organic solvent.

More particularly, in some embodiments, the presently disclosed method comprises mixing a small amount of a sample known or suspected of containing PTC (e.g., residual PTC from a radiosynthesis) with a small amount of an aqueous potassium or sodium salt solution (e.g., a potassium or sodium salt solution comprising a chromophoric ion). In some embodiments, the sample is from a radiotracer or radiopharmaceutical dose. In some embodiments, the radiotracer or radiopharmaceutical comprises a radioisotope selected from the group including, but not limited to, ¹⁸F, ¹¹C, ¹³N, ¹⁵C, ³²P, ⁶⁷Ga, ^{99m}Tc, and ¹²³I. In some embodiments, the radiotracer or radiopharmaceutical comprises ¹⁸F. In some embodiments, the sample comprises an aliquot from a radiopharmaceutical dose and the radiopharmaceutical is selected from the group comprising [¹⁸F]2-fluoro-2-deoxy-D-glucose (FDG) [¹⁸F]sodium fluoride; [¹⁸F]3′-deoxy-3′-fluorothymidine (FLT), [¹⁸F]fluoromisonidazole, [¹⁸F]florbetaben, [¹⁸F]florbetapir, [¹⁸F]fluoro-ethyl-tyrosine (FET), [¹⁸F]flutemetamol, [¹⁸F]-fluorocholine (FCH), [¹⁸F]fluoroethylcholine (FECH), [¹⁸F]fallypride, and [¹⁸F]6-fluor-L-2,3-dihydroxyphenylalanine (FDOPA).

In some embodiments, the small amount of the sample (e.g., the aliquot of the radiopharmaceutical or radiotracer) has a volume of between about 50 microliters (μL) and about 100 μL (e.g., 50, 55, 60, 65, 70, 75, 80, 85, 90, 95, or about 100 μL). In some embodiments, the amount of sample is about 50 μL.

The amount of the aqueous salt solution can vary depending upon the concentration of the salt solution and/or the identity of the salt in the aqueous salt solution. In some embodiments, the concentration of salt is optimized so that the volume of the aqueous salt solution is between about 0.5 and about 1 times the volume of the radiotracer or radiopharmaceutical dose amount. In some embodiments, the amount of the aqueous salt solution is about 50 μL.

In some embodiments, the aqueous salt solution comprises at least one potassium or sodium salt of a chromophoric anion. In some embodiments, the aqueous salt solution comprises potassium permanganate (KMnO₄) or sodium resazurin. However, any suitable potassium or sodium salt of a chromophoric anion can be used. In some embodiments, the chromophoric anion is a carboxylate or sulfonate of an organic compound that includes a chromophoric group selected from an anthraquinone, a methine, a phthalocyanine, a nitro group, an azo group, and a triarylmethane. Thus, for example, the potassium or sodium salt can be a sodium or potassium salt of a compound known in the art for use as a dye, such as a diazo dye or an anthraquinone dye. In some embodiments, the salt is a potassium or sodium salt of resazurin or a resazurin analog, e.g., a compound wherein the hydroxyl group of the resazurin is replaced by an alkyl or aryl ether.

In some embodiments, the aqueous salt solution comprises potassium permanganate at a concentration of between about 0.05 and about 0.5 M (i.e., about 0.05, 0.10, 0.15, 0.20, 0.25, 0.30, 0.35, 0.40, 0.45, or about 0.50 M). In some embodiments, the concentration of the potassium permanganate is about 0.2 M.

In some embodiments, the aqueous salt solution comprises a mixture of sodium resazurin and potassium carbonate. The concentrations of the sodium resazurin and the potassium carbonate can each be between about 0.01 M and about 0.06 M. In some embodiments, the ratio of the concentrations of the sodium resazurin and the potassium carbonate can be between about 5:1 and about 1:2. In some embodiments, the ratio of the concentrations can be between about 1.2:1 and about 1:1.2. In some embodiments, the sodium resazurin and the potassium carbonate are present at the same concentration. In some embodiments, the concentration of the sodium resazurin is about 0.04 M or about 0.05 M. In some embodiments, the concentration of the potassium carbonate is about 0.04 M or about 0.05 M.

The sample and the aqueous salt solution can be mixed in any suitable container. In some embodiments, the small amount of sample (e.g., the aliquot of radiotracer/radiopharmaceutical dose) and the salt solution can be mixed in a centrifuge tube (e.g., a 1.5 mL polypropylene centrifuge tube). Then, a suitable organic solvent is added to form a biphasic mixture. The amount of organic solvent added can range from about 10 to about 20 times the volume of the small amount of sample. In some embodiments, the amount of organic solvent is between about 0.5 mL and about 1.4 mL. In some embodiments, the amount of organic solvent is about 1 mL.

Suitable organic solvents include those that are immiscible in water (e.g., aromatic solvents, such as benzene and toluene; halogenated solvents, such as dichloromethane (DCM), chloroform, carbon tetrachloride, 1,2-dichloroethane, and trichloroethylene; alkanes such as pentane, hexane, cyclohexane, and heptane; certain esters, such as ethyl acetate and butyl acetate; certain ethers, such as diisopropyl ether, diethyl ether, and methyl-t-butyl ether; and certain ketones and alcohols, such as 2-butanone and n-butanol). In some embodiments, the organic solvent should also be one that does not absorb light at the same wavelength as an absorption maximum of the chromophoric moiety of the salt of the aqueous salt solution (i.e., a “region of interest” of the chromophoric moiety via UV-Vis spectroscopy). For example, if the chromophoric moiety of the salt absorbs light at 530-570 nm (e.g., 532 nm), a suitable organic solvent would be a solvent that does not absorb at 530-570 nm (e.g., 532 nm) significantly. In some embodiments, the organic solvent is chloroform or DCM. In some embodiments, the organic solvent is DCM.

After the organic solvent is added, the biphasic mixture is mixed so that at least some of a complex formed between any PTC present in the sample and the salt from the aqueous salt solution is extracted into the organic phase. The mixing can include any suitable mixing method, e.g., stirring, shaking, sonicating, or vortexing. In some embodiments, the mixing comprises vortexing. In some embodiments, the vortexing is performed for about 30 seconds. After the mixing, the mixture is given time for the two phases to completely separate. In some embodiments, the mixture is allowed to separate for about 1 minute.

As noted above, at this point, some of a complex formed between any PTC present in the sample and the salt from the aqueous salt solution will be dissolved in the organic phase. Thus, the organic phase can be analyzed to detect the presence and/or concentration of the complex between the PTC and the salt. The amount of complex present in the organic phase will be in proportion to the amount of residual PTC present in the sample. Thus, the concentration of complex in the organic phase is indicative of the amount of residual PTC in the sample.

The organic phase can be analyzed via any suitable method. If the salt in the aqueous salt solution includes a chromophoric anion, some of the chromophoric anion will now be present in the organic phase. More particularly, the amount of the chromophoric anion in the organic phase will be generally proportional to the amount of residual PTC in the sample. Thus, for instance, analyzing the organic phase can comprise visually comparing the organic phase to a standard solution (e.g., a solution comprising a known amount of the PTC/salt complex or an organic phase extracted from a mixture of the salt and a sample comprising a known amount of the PTC) or measuring the light absorbance of the organic phase at one or more wavelengths of interest (i.e., one or more wavelengths that correspond to a wavelength absorbed by the chromophoric anion of the salt of the aqueous salt solution) and comparing the light absorbance of the organic phase to the light absorbance of one or more standard solutions, each comprising a known concentration of the complex between the PTC and the salt of the aqueous salt solution or comprising the organic phase extracted from an aqueous mixture of the salt and a known amount of the PTC. In particular, the speed and accuracy of spectrophotoscopic analysis in combination with the ready availability of simple devices for making visible spectrophotometric measurements can provide for the measurement of residual PTC in radiotracer or radiopharmaceutical dose formulations easily at the point of care (e.g., in a hospital or clinic) or synthesis (e.g., at a radiosynthesis laboratory or manufacturing site).

More particularly, the relationship between light absorption and analyte concentration can be given by Beer's Law:

A=εlc

where A is absorbance, ε is the molar attenuation coefficient (also referred to as the extinction coefficient) of the absorbing species, c is the concentration of the absorbing species in moles per liter and l is the optical path length in centimeters. The light absorption of the organic phase can be detected using a spectrophotometric device, such as a UV-Vis spectrophotometer, which can measure the intensity of light passing through a sample, which can also be referred to as transmittance, which is expressed as a percentage. Transmittance can be converted to absorbance via the formula:

A=−log(% T/100%),

where A is absorbance, and % T is % transmittance. While absorbance is generally expected to have a linear relationship to concentration, in some embodiments, equilibrium effects can affect the results.

Devices for measuring absorption, such as UV-Vis spectrophotometers, generally include at least a light source, a light detector, and a holder or reservoir for the sample being analyzed. If the light source emits light at multiple wavelengths (e.g., if the light source is a Xenon arc lamp), the device can further include a diffraction grating or prism to separate the light so that only a select wavelength reaches the sample holder. Other suitable light sources include lasers and LEDs that can emit light at select wavelengths of interest. Suitable detectors include photomultiplier tubes, photodiodes, photodiode arrays, and charge-coupled devices (CCDs). Suitable holders/reservoirs include glass or quartz cuvettes.

In an exemplary embodiment of the presently disclosed subject matter, when the aqueous salt solution comprises KMnO₄ or sodium resazurin, the organic layer can be analyzed using a spectrophotometric device that can illuminate the organic layer at 532 nm. Thus, in some embodiments, the spectrophotometric device can use a green light source (e.g., a 5 milliwatt green laser or light-emitting diode (LED)) to generate the 532 nm light. However, if the aqueous salt solution comprises a salt other than KMnO₄ or sodium resazurin and the chromophoric anion of the salt does not absorb green light, light of another wavelength or color can be used to illuminate the sample and the device can include another light source (e.g., a LED or a laser that emits light of a color other than green). The device can also include one or more additional components selected from the group including, but not limited to, a sample reservoir for the organic phase (e.g., a glass cuvette), a solid body for holding the sample reservoir in the path of a light beam from the green laser or other light source, a microprocessor, a power source, and a display (e.g., a computer screen or display screen) for displaying one or more absorbance measurement values. Thus, in some embodiments, the presently disclosed methods can be performed using small, inexpensive and portable spectrophotometric devices.

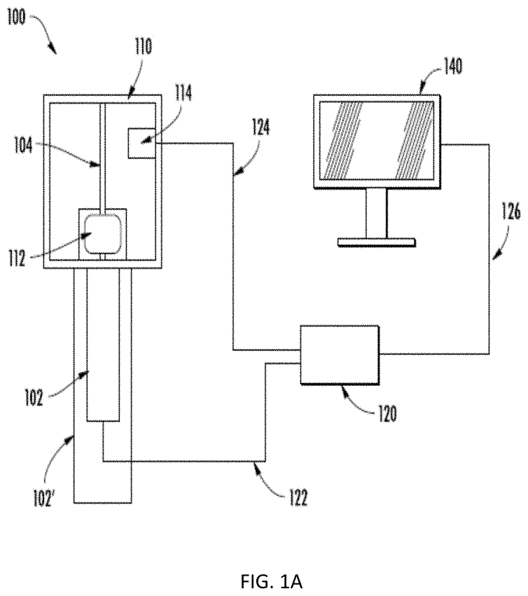

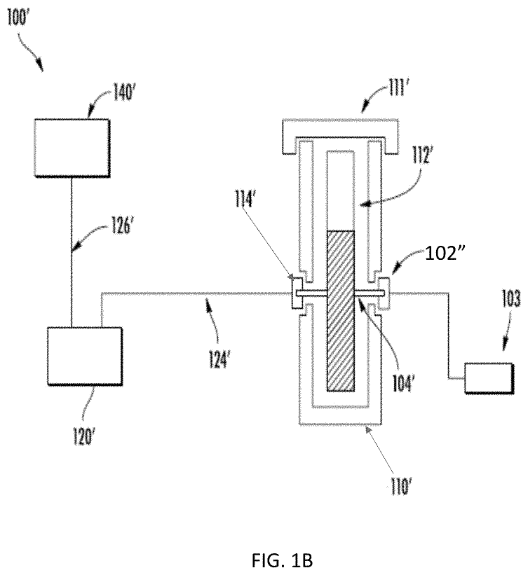

FIG. 1A shows a top view of an exemplary device 100 for use in analyzing the organic phase if the salt of the aqueous salt solution comprises a chromophoric moiety. Device 100 includes a solid plastic body 110 where sample holder 112 (e.g., a standard-sized glass or quartz cuvette) can be positioned in the path of light beam 104 from light source 102 (e.g., a green laser or LED emitter) housed in housing 102′. Body 110 can be, for example a 3D printed plastic body. The light that is not absorbed by the sample in cuvette 112 (i.e., the transmitted light) can be detected by light detector 114. The top of body 110 can have an opening so that sample holder 112 can be removed, but which can be covered by a lid to block ambient light during measurements. Light source 102 can be controlled via microprocessor 120 (e.g., a Raspberry Pi) which can also receive data from light detector 114 and process the data. Microprocessor 120 is connected to light source 102 via wiring 122 and to light detector 114 via wiring 124. In some embodiments, microprocessor 120 can also display and/or store data. For example, microprocessor 120 can store calibration curve data and compare new sample data to the calibration curve. Calibration curve data can be obtained from one or more standard solutions of the salt complex of the PTC of interest, e.g., where the PTC concentrations of the standard solutions can center on or include the highest acceptable concentration of that PTC. As shown in FIG. 1A, microprocessor 120 can also be connected to a display screen 140 (e.g., a video display) via wiring 126. If desired, one or more of wiring 122, 124, and 126 can be replaced by a wireless receiver and or transmitter for wireless communication.

FIG. 1B shows a side view of exemplary device 100′, which is similar to device 100 of FIG. 1A. Device 100′ includes body 110′, shown covered with top 111′ to prevent ambient light entry into body 110′. Cuvette 112′ is placed inside body 110′ such that light beam 104′ from light source 102″ (e.g., a green laser or LED) passes through a sample in cuvette 112′. Light source 102″ is connected to power source 103. Detector 114′ detects the amount of light from light beam 104′ that passes through the sample and then passes that information (via wiring 124′) to microprocessor 120′ (e.g., a Raspberry Pi) to process the data. The processed data can be transmitted to display 140′ via wiring 126′. Alternatively, wiring 126′ can be absent and microprocessor 120′ can communicate with display 140′ wirelessly using a wireless transceiver (e.g., Bluetooth).

Alternatively, since the extracted PTC/salt complex is soluble in nonconductive organic solvents and contains an organic salt compound, it is also possible to measure the PTC concentration by electrical resistance. The presence of charged ions in a normally nonconductive organic solution provides for the solution to conduct electricity, analogous to when water (a nonconductor) becomes electrically conductive when it contains an ionic solute. Thus, in some embodiments, the presently disclosed subject matter provides a method of detecting or quantifying PTC wherein the analysis of the organic phase comprises measuring the electrical conductivity (or resistance) of the organic phase and comparing the electrical conductivity (or resistance) of the organic phase to one or more standard solutions comprising a known concentration of a complex between the PTC and the salt of the aqueous salt solution or the organic extract from an aqueous mixture comprising the salt and a known concentration of the PTC. In some embodiments, the measuring is performed with an electrical test cell. The electrical test cell can comprise a device suitable for measuring resistance, e.g., an ohmmeter, a multimeter (e.g., a digital multimeter), or a LCR meter.

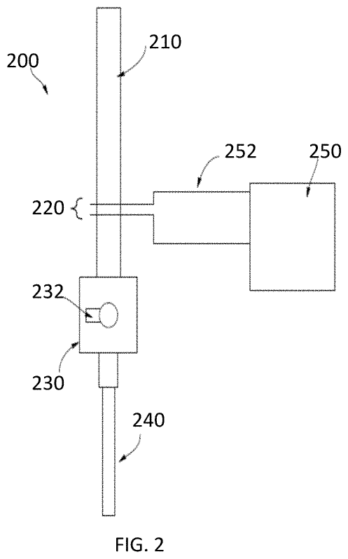

FIG. 2 shows a simple exemplary device 200 that can be used to measure the resistance of an organic phase of the presently disclosed methods. Device 200 can include sample tube 210 (e.g., a polypropylene tube) where the organic phase being analyzed can be placed. The body of tube 210 also contains two electrodes 220 (e.g., two stainless steel electrodes) spaced between about 2 and about 3 mm apart. Electrodes 220 are connected to microprocessor 250 (e.g. a Raspberry Pi or digital multimeter) via wiring 252. Microprocessor 250 can be configured to analyze and display data. Body 210 is also attached to a 2-way valve 230, which includes actuator 232 to control the flow of a sample from tube 210 to waste drain line 240. During use of the device, resistance through the organic phase (present in sample tube 210) can be measured for a short time (e.g., about 10 seconds). In some embodiments, the average value of multiple measurements can be obtained. In some embodiments, the average of two or three separate test samples (i.e., the average of the resistance of the organic phases obtained from two or three separate aliquots of the radiotracer or radiopharmaceutical dose) can be used to provide the PTC concentration in the original sample containing or suspected of containing PTC.

In some embodiments, the presently disclosed subject matter provides a method of conducting a QC test on a radiopharmaceutical or radiotracer, wherein the method comprises: a) mixing an aliquot of a radiopharmaceutical solution with an aqueous solution comprising a potassium and/or sodium salt to provide an aqueous mixture; b) adding an organic solvent to the aqueous mixture to provide a biphasic mixture comprising an aqueous phase and an organic phase; c) mixing the biphasic mixture for a period of time; d) separating the organic phase from the aqueous phase; and e) analyzing the organic phase, thereby determining the concentration of a residual phase transfer catalyst (PTC) in the radiopharmaceutical or radiotracer. In some embodiments, the analyzing comprises (i) measuring an optical absorbance of the organic phase or an electrical conductivity of the organic phase, and (ii) comparing the optical absorbance or electrical conductivity to an optical absorbance or electrical conductivity of one or more standard solutions, wherein each of the one or more standard solutions comprises a known concentration of the PTC complexed to the potassium and/or sodium salt or an organic extract from an aqueous mixture of the potassium and/or sodium salt and a known concentration of the PTC.

In some embodiments, the residual PTC is K-222, TBAHC, or 18-crown-6. In some embodiments, the radiopharmaceutical is selected from the group consisting of [¹⁸F]2-fluoro-2-deoxy-D-glucose (FDG) [¹⁸F]sodium fluoride; [¹⁸F]3′-deoxy-3′-fluorothymidine (FLT), [¹⁸F]fluoromisonidazole, [¹⁸F]florbetaben, [¹⁸F]florbetapir, [¹⁸F]fluoro-ethyl-tyrosine (FET), [¹⁸F]flutemetamol, [¹⁸F]-fluorocholine (FCH), [¹⁸F]fluoroethylcholine (FECH), [¹⁸F]fallypride, and [¹⁸F]6-fluor-L-2,3-dihydroxyphenylalanine (FDOPA). In some embodiments, the potassium and/or sodium salt is KMnO₄ or a mixture of sodium resazurin and potassium carbonate.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

### QC Using Potassium Permanganate

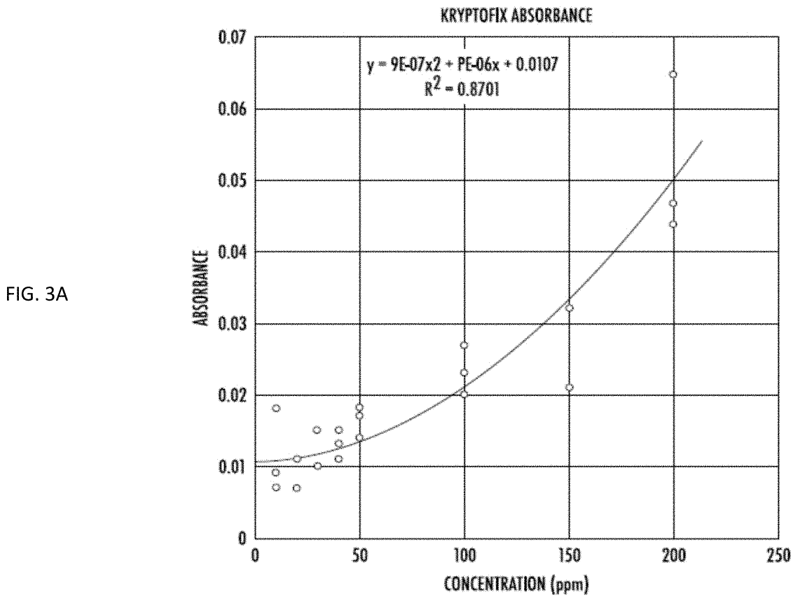

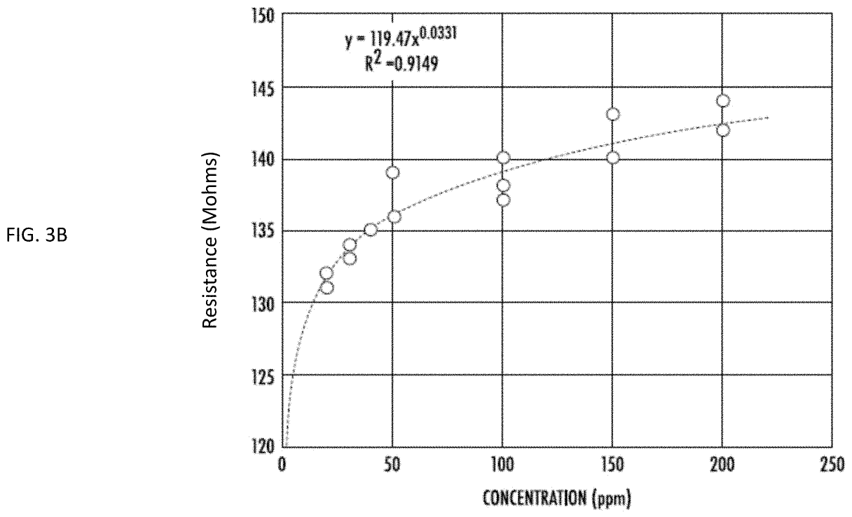

A small amount (50-100 μL) of each solution of a series of simulated radiotracer dose solutions comprising known concentrations (between about 20 and 250 ppm) of K-222 was mixed with a small amount (50 μL) of a highly colored aqueous salt solution containing 0.2 M potassium permanganate in a 1.5 mL polypropylene tube. Then, dichloromethane (DCM, 1 mL) was added to the tube and the contents were mixed by vortexing for 30 seconds. The two phases were given time to separate (about 1 minute). At this point, the DCM layer contains some of a PTC-complex comprising the chromophoric potassium permanganate. The DCM layer was then analyzed on a spectrophotometric device that used a green (532 nm) five-milliwatt laser as the light source. The PTC/chromophore complex absorbed the 532 nm light, and the amount of absorbance was proportional to the amount of PTC present. As the PTC content of the simulated radiotracer dose increased, the amount of light transmitted to the light detector decreased due to increased absorption. See FIG. 3A.

Similarly, a small amount (50-100 μL) of each of a series of simulated radiotracer dose solutions comprising known concentrations of 18-crown-6 was mixed with a small amount (50 μL) of the aqueous salt solution containing 0.2 M potassium permanganate in a 1.5 mL polypropylene tube. Then, DCM (1 mL) was added to the tube and the contents were mixed by vortexing for 30 seconds. The two phases were given time to separate (about 1 minute). The DCM layer was analyzed suing a photodiode resistor. The resistance outputs are shown in FIG. 3B.

### Example 2

### QC Using Sodium Resazurin/Potassium Carbonate

### Materials and Methods:

Certified American Chemical Society grade dichloromethane (DCM) and trichloromethane (chloroform), 1,4,7,10,13,16-hexaoxacyclooctadecane (18-crown-6), 4,7,13,16,21,24-Hexaoxa-1,10-diazabicyclo[8.8.8]hexacosane (K-222), dyes, potassium carbonate, tetrabutylammonium hydroxide solution, 1.5 mL Eppendorf centrifuge tubes, disposable micropipette tips, micropipettes, black cuvettes, a digital multimeter (Fluke 115), and solid phase extraction cartridges were purchased from Fisher Scientific (ThermoFisher Scientific, Waltham, Mass., United States of America). [¹⁸F]fluoride was obtained from PETNET Solutions (Knoxville, Tenn., United States of America).

A simple spectrophotometer similar to that shown in FIGS. 1A and 1B was made by placing a light detector (Adafruit TSL-2591, Adafruit Industries, New York, New York, United States of America) inside of a semi-reflective polylactic acid 3-D printed (Ultimaker-2 Extended+ 3-D Printer, MatterHackers, Foothill Ranch, Calif., United States of America) body. A cuvette holder with a hole to admit laser light was integrated into the front side. A horizontal trough-shaped portion was incorporated to hold the laser. A top cover was also made for use during data acquisition. The detector was connected to a Raspberry Pi microprocessor (Raspberry Pi Foundation, Cambridge, United Kingdom). The 532 nm laser (Light Vision JPM-5-3 532-nm Laser Module (LightVision Technologies, Corp., Gueishan Township, Taiwan; output 3.5 mW/Output stability ±15% at 25° C.) was powered by the Raspberry Pi microprocessor (Raspberry Pi Foundation, Cambridge, United Kingdom). Data was displayed on an attached computer monitor.

Before each sample measurement, the laser output was determined and adjusted without a cuvette. Starting with the laser at room temperature (22° C.), repeated intensity measurements were taken until values reaches 145±3 arbitrary units of light intensity (the laser output decreases as the laser heats with use). During acquisition, a new light intensity value is displayed every 2 s. Each reported measurement comprises the average of four of these values (total measurement time is 8 s). This method allowed the laser output to be consistent during sample measurement. Alternatively, the laser adjustment can be made with a cuvette in place to a value of 120±3. After laser adjustment, the pre-made sample extract was promptly pipetted into the cuvette, and four transmittance values are taken (total acquisition time 8 s). The average of these four values was taken as the final measurement value. After measuring several samples, it was observed that allowing the laser to cool (e.g., at least for 12 minutes between uses for the particular laser used herein) can be beneficial.

TBAHC was made starting with 50.51 mL (50 g) of 40-wt % (1.5 M) tetrabutylammonium hydroxide by rapidly bubbling gaseous carbon dioxide through the solution with stirring for 8 hours until the conversion was complete. Stock solutions were made by dissolving 30 mg of PTC in a 100±0.08 mL volumetric flask in saline and filling to the calibration line. Serial dilutions were then made at 0, 12, 25, 50, 75, 100, and 150 ppm by adding appropriate amounts of the stock solution to 10±0.02 mL volumetric flasks and diluting to the calibration mark with saline. In the case of TBAHC, 77.1 μL of the solution was added to a 10±0.02 mL volumetric flask and diluted to the calibration mark with saline.

Sodium salt test solutions of phenolic and acid dyes were made for initial testing by reacting with a stoichiometric quantity of sodium hydroxide to produce a 0.01 M solution.

7-Hydroxy-3H-phenoxazin-3-one-10-oxide sodium salt (resazurin) test solutions of different concentrations and ratios of resazurin to carbonate were made by dissolving varying amounts sodium resazurin and potassium carbonate in 10±0.02 mL volumetric flasks with distilled water. The flasks were vortexed for several minutes, and then filled to the calibration mark with distilled water.

The PTC sample extraction method uses 50-100 μL of the aqueous PTC solution (or radiotracer dose solution) with 50 μL of the resazurin test solution. These solutions were combined in a polypropylene 1.5 mL centrifuge tube, and 1 mL of DCM was added. The mixture was vortexed on high for 30 s, and the layers are allowed to separate. The DCM layer (800 μL) was then removed for analysis using a micropipette.

An electrical test cell similar to that shown in FIG. 2 was constructed from a 1 mL polypropylene syringe body attached to a 2-way valve with a line to drain out the waste. Two perpendicular 16-gauge needles spaced 2.5 mm apart were place through the syringe body at the 0.5 mL mark. Then, the electrodes were attached to the digital multimeter electrical leads. To acquire a measurement the sample was pipetted into the syringe body, and when done the waste was drained out the bottom. In order to obtain electrical resistance measurements, the digital multimeter was turned to the Ohms setting. Next, the meter was set to take the average value. At 10 s of acquisition the “capture-value” button was selected, the average value was displayed on the LCD screen.

[¹⁸F]Fluorocholine radiosyntheses were carried out using a Sofie-Elixys/Flex-Chem automated radiosynthesis platform (Sofie Biosciences, Culver City, Calif., United States of America) according to a published procedure, with the only deviation being the substitution of 30 mg of 8-crown-6 for K-222 [15].

### Results:

Mixing the resazurin test solution with a dilute aqueous solution of PTC in DCM or chloroform resulted extraction of the blue dye into the organic layer.

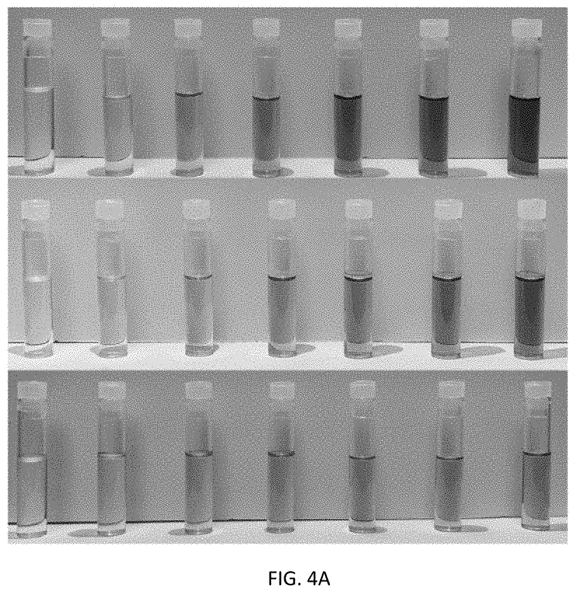

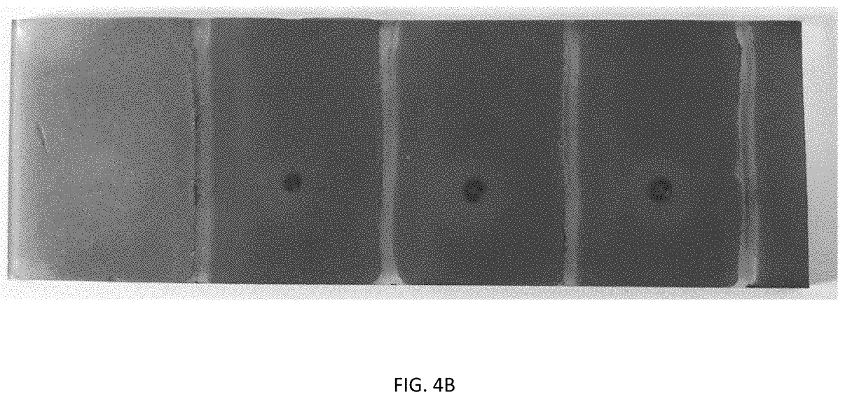

Without the addition of the PTC solution, no blue coloration was observed in the organic solvent. DCM was used as the organic solvent for further measurement studies. The DCM layer extractions of the PTC standards showed that the varying dye concentrations were easily visualized colorimetrically. At 0 ppm, the aqueous dye imparts a very faint pink color to the DCM layer. The 6 ppm extraction appears identical to the 0 ppm. At 12 ppm, the DCM layer has a faint blue color. As the PTC concentration of the standard solution increased, the DCM layer became progressively darker. This was true for each of the PTCs tested. See FIG. 4A. For comparison, the standard iodoplatinate staining results are shown in FIG. 4B.

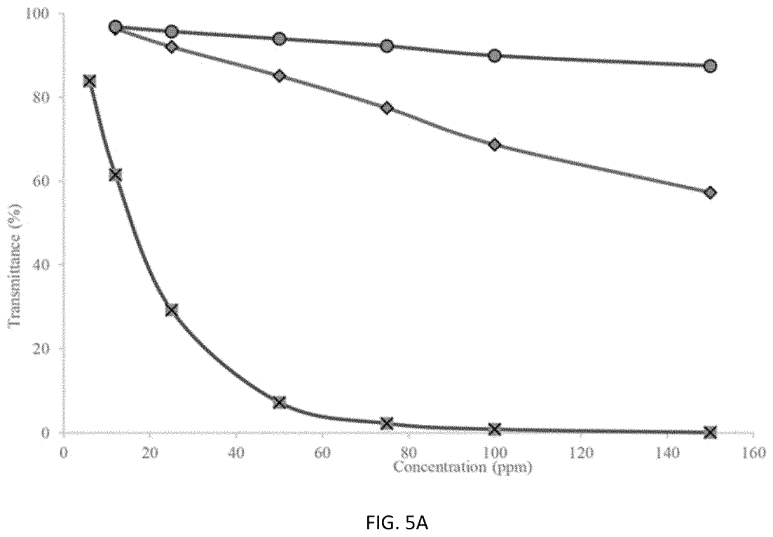

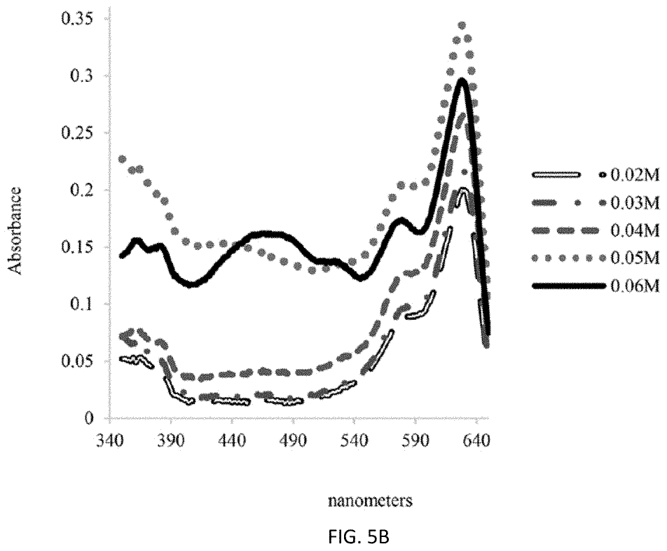

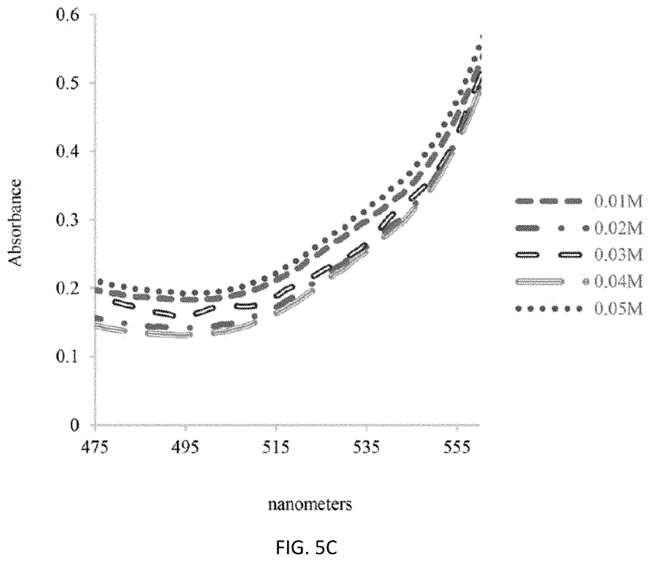

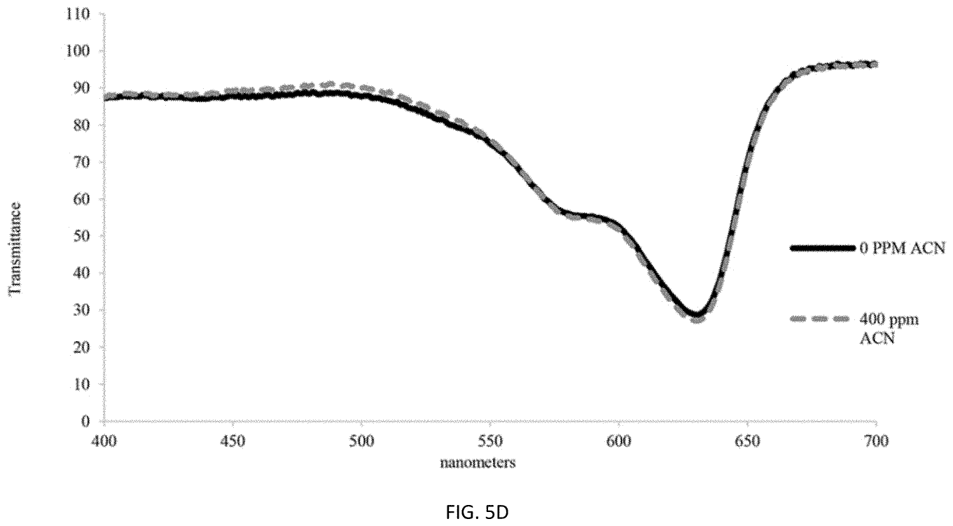

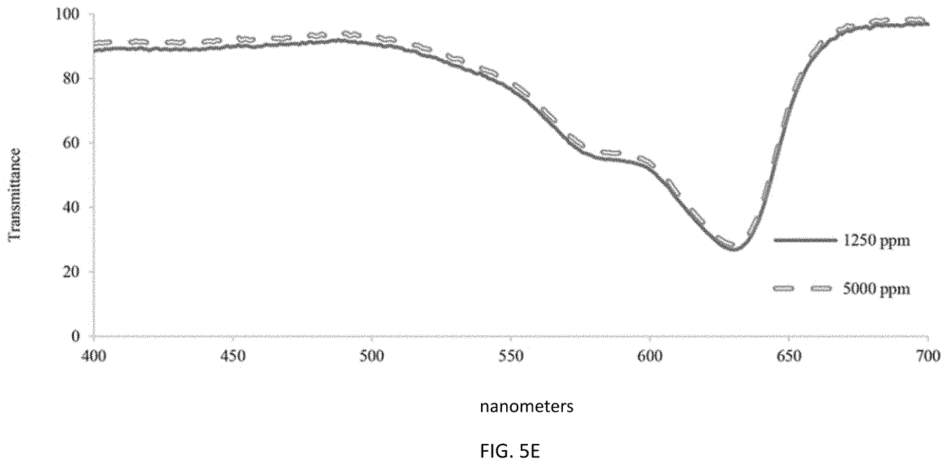

Transmittance measurements at 532 nm were taken of the PTC-resazurin complex extracts in DCM of K-222, 18-crown-6, and TBA using a UV-Vis spectrophotometer. Each PTC transmittance value was measured at 0, 6, 12, 25, 50, 75, 100, and 150-ppm. See FIG. 5A.

The sodium resazurin and potassium carbonate concentrations of the test solution were optimized using a UV-Vis spectrophotometer. First, sodium resazurin and equimolar potassium carbonate were tested at 0.02, 0.03, 0.04, 0.05, and 0.06 M with a 100 ppm K-222 solution. See FIG. 5B. It was found that the 0.05 M sodium resazurin/0.05 potassium carbonate showed the highest absorbance at 532 nm. Next, the amount of potassium carbonate was varied from 0.01-0.05 M at a constant 0.05 M resazurin concentration. See FIG. 5C. The 0.05 M resazurin/0.05 M potassium carbonate solution showed the highest absorption

To serve as a useful methodology for residual PTC testing of radiopharmaceuticals, the presence of residual solvents up to the USP limit should have no significant effect on the measurement obtained. To establish whether residual solvents pose an interference problem, a 50 ppm K-222 concentration was tested that contained varying amounts of acetonitrile or ethanol. The presence of either acetonitrile (up to 400 ppm) or ethanol (up to 5000 ppm) had no effect on the transmittance values obtained. See FIGS. 5D and 5E.

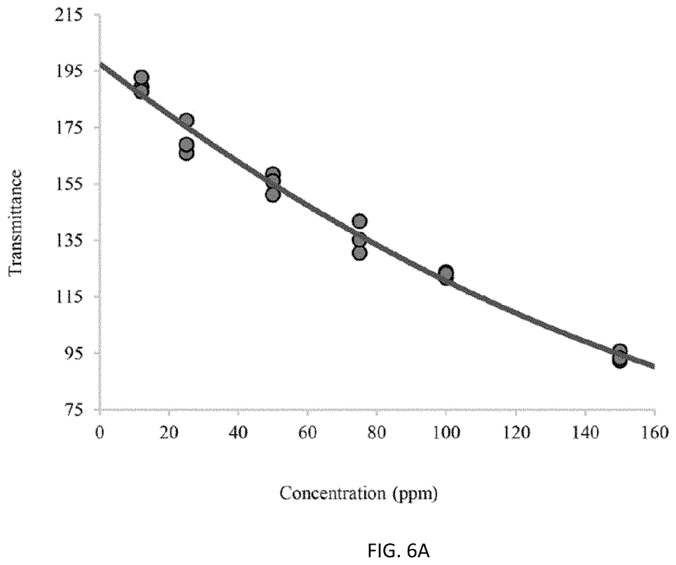

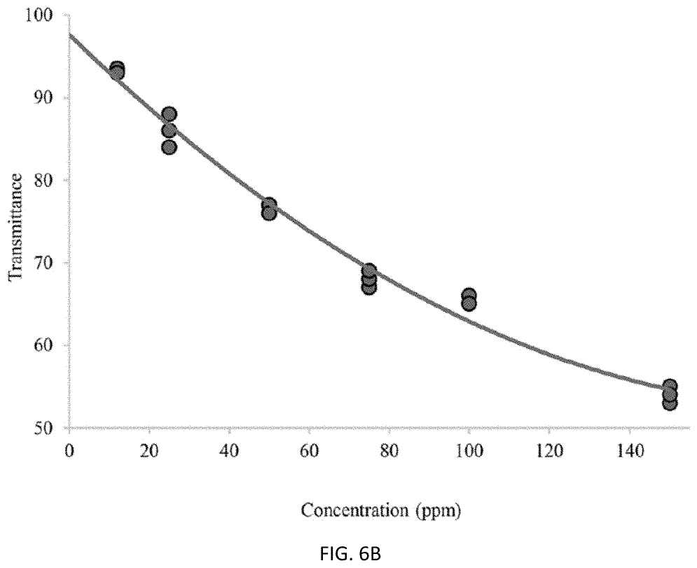

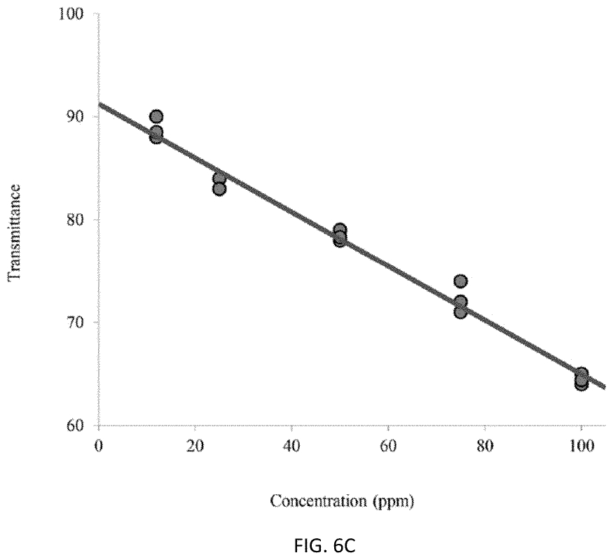

Calibration curves for K-222, TBA, and 18-crown-6 transmittance values were generated using the in-house built spectrophotometer prototype with values ranging from 0-150 ppm (n=3 at each concentration) with each point being the average of four sequential measurements of the same sample (as described above). See FIGS. 6A-6C. The lower limit of quantitation was determined to be 12 ppm for each PTC tested. 18-crown-6 concentrations could not be accurately measured above 100 ppm. Without being bound to any one theory, this finding is believed most likely due to near saturation of the DCM with the resazurin-PTC complex at just above 100 ppm. In the case of TBA, the interior of the spectrophotometric device was lined with a layer of highly reflective aluminum to increase the light reaching the detector; otherwise, measurements at the 150-ppm concentration could not be made due to the low light level reaching the detector.

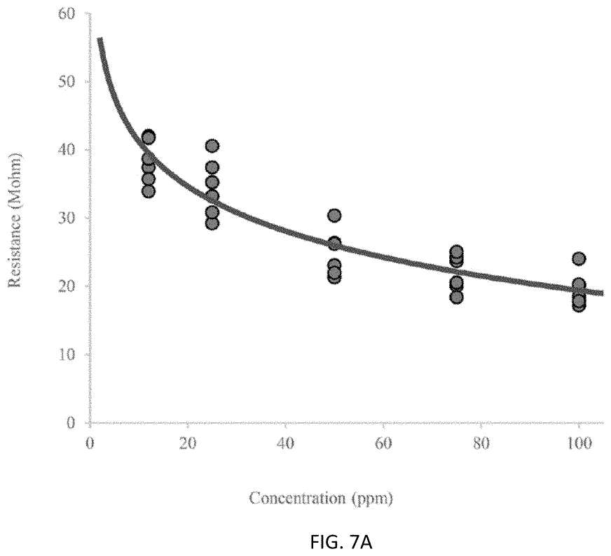

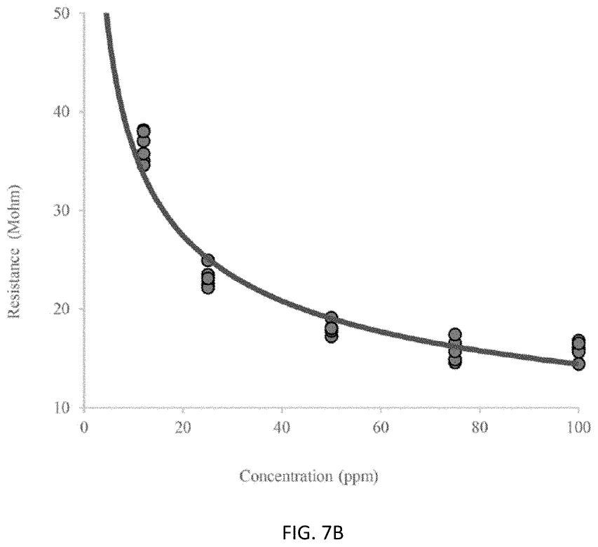

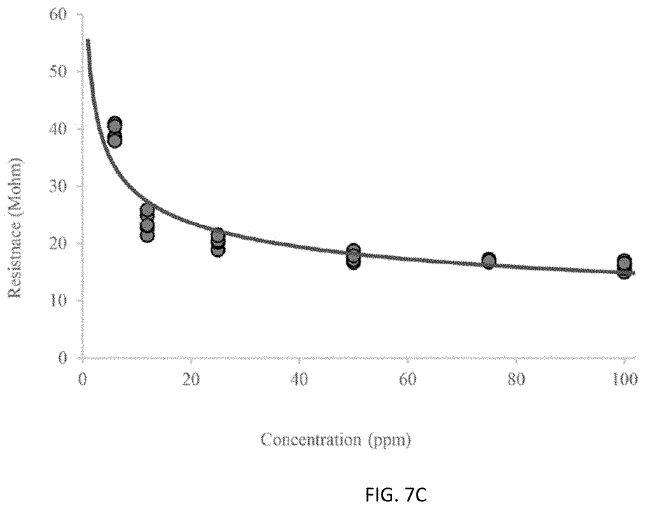

The electrical resistance of PTC standards ranging from 0-100 ppm was measured using the simple electrical test cell described above, and calibration curves for each PTC were made (n=6 for each concentration). See FIGS. 7A-7C. PTC concentrations could be accurately measured by electrical conductance for samples up to about 100 ppm. Radiopharmaceutical sample measurements were made by taking the average of the resistance values for two different samples. As with the transmittance measurements, ACN and ethanol up to the USP limits did not affect the measurement outcomes.

Three commercially prepared [¹⁸F]2-fluorodeoxyglucose dose samples were each analyzed for residual K-222 by visual colorimetry, spectrophotometrically, and by electrical conductance. Results are summarized below in Table 1.

The resazurin based colorimetry technique was used in the synthesis of [¹⁸F]Fluorocholine to validate the effectiveness of the washing protocol for removing 18-crown-6 from the cation exchange solid-phase-extraction cartridge used to trap the product. The published procedure, originally developed to remove the reactant N,N-dimethylaminoethanol and K-222, removed essentially all 18-crown-6 from the radiotracer dose. The DCM extract of the final [¹⁸F]Fluorocholine dose was identical to the 0 ppm standard.

TABLE 1
Measured Values of Residual K-222 in Three Separate Commercially
Produced [18F]-2-fluorodeoxyglucoes does samples.

| | | Transmittance | |
|---|---|---|---|
| | | (arbitrary units of | |
| | Visual | light | Resistance |
| [¹⁸F]FDG Sample | Colorimetry | intensity)/ppm | (Mohm)/ppm* |
| 1 | 0-<12 | 96/0 | >60/0 |
| 2 | 0-<12 | 97/0 | >60/0 |
| 3 | 0-<12 | 97.8/0 | >60/0 |

*Resistance measurement above 60 Mohm exceed the limits of the multimeter.

### Discussion:

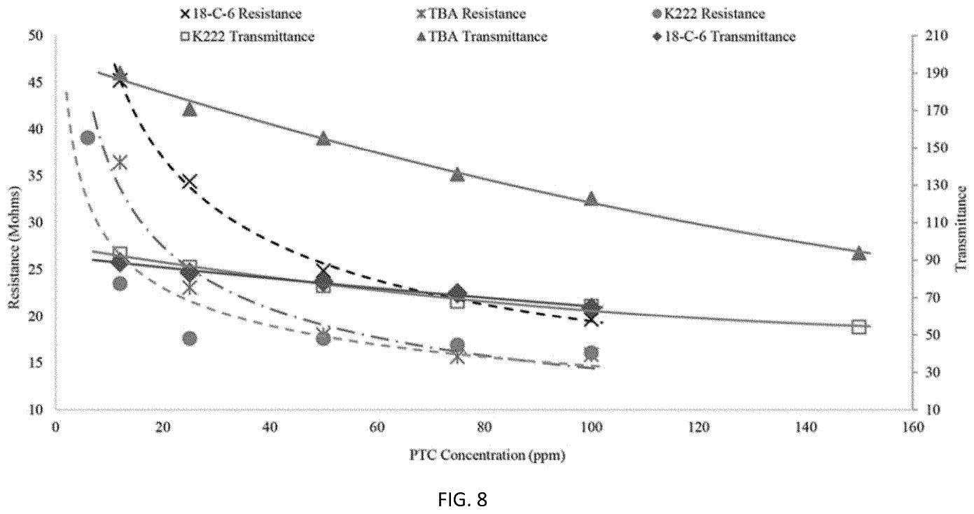

Each PTC exhibited lower transmittance values with increasing PTC concentration of the sample. See FIG. 8. TBA sample extracts showed the strongest light absorption and 18-crown-6 the least. For each PTC tested, the best-fit-line is a nonlinear polynomial function; for 18-crown-6 the line appears nearly linear. The resistance calibrations curves also have nonlinear best-fit-lines that are power functions. In agreement with the transmittance measurements, the 18-crown-6 best-fit-line exhibits higher linearity.

Residual PTC in three commercially produced [¹⁸F]2-fluorodeoxyglucose radiopharmaceutical doses was successfully measured using the optimized resazurin dye test solution. The testing of three separate commercially produced dose samples for K-222 confirmed the absence of residual K-222 by visual colorimetry, spectrophotometrically, and by conductance. Additionally, resazurin-based colorimetry quickly verified the absence of 18-crown-6 in [¹⁸F]fluorocholine preclinical doses.

Resazurin based visual colorimetry is significantly more quantitative than the current spot-test method. Quantitative spectrophotometric measurements were made in the 0-100 ppm range (18-crown-6) and 0-150 ppm range (K-222 or TBA). The ability to use a low cost 532 nm laser source and an inexpensive detector to obtain accurate quantification of PTC concentrations is highly amenable for development into an inexpensive QC device that can provide an automated electronic output for batch reports, or for integration into a more complex QC testing platform that will be able to analyze for any PTC. Although quantitative results are not currently required by radiopharmaceutical standards, the industry is always growing to adjust standards to improve quality control and efficiency. A more automated, quantitative platform could be used to monitor variability in residual PTC across batches to hone production conditions for more consistent products, and to potentially find flaws in production methods that lead to large inconsistencies.

Measuring electrical resistance of the PTC-resazurin complex in organic solution is also a viable PTC analysis method that allowed accurate quantification in the 0-100 ppm range (see FIG. 8), but averaging two separate sample measurements can make it somewhat less attractive. Further optimization of the method could potentially improve the accuracy so that only a single sample analysis is needed. The evaluation of the effect of expected interferents such as acetonitrile and ethanol on the measurements revealed that these methods of measurement are not affected by residual solvent, and are therefore valid methods suitable for use in a radiopharmacy.

### REFERENCES

All references listed below, as well as all references cited in the instant disclosure, including but not limited to all patents, patent applications and publications thereof, and scientific journal articles, are incorporated herein by reference in their entireties to the extent that they supplement, explain, provide a background for, or teach methodology, techniques, and/or compositions employed herein.
[1] Tabeta R, Saitô H. A High-resolution ¹³ C NMR Study of Some Ethylene Oxide Oligomers, Cryptands, and Crown Ethers Complexed with a Variety of Metal Ions in the Solid State. Conformational Change of Ligand Molecules as Viewed from the Conformation-dependent Displacements of ¹³C Chemical Shifts. Bull Chem Soc Jpn. 1985; 58:3215-3224.
[2] Letcher, T M, Mercer-Chalmers J D, Kay R L. Volume changes in complex formation between crown ethers of cryptand-222 and alkali metals in various solvents. Pure Appl Chem. 1994; 66:419-427.
[3] Buschmann H J, Mutihac R C, Schollmeyer E. Complex formation of crown ethers and cryptands with alkali metal and ammonium ions in chloroform. J Solution Chem. 2009; 38:209-217.
[4] Poonia N S, Bajaj A V. Coordination chemistry of alkali and alkaline earth cations. Chem Rev. 1979; 79:389-445.
[5] Adams D, Clark J. Nucleophilic routes to selectively fluorinated aromatics. Chem Soc Rev. 1999; 28:225-231.
[6] Jacobson O, Kiesewetter D O, Chen X. (2014). Fluorine-18 radiochemistry, labeling strategies and synthetic routes. Bioconjug chem. 2014:26:1-18.
[7] Culbert P A, Adam M J, Hurtado E T, Huser J M A, Jivan S, Lu J, Zeisler, S K. (1995). Automated synthesis of [¹⁸F] FDG using tetrabutylammonium bicarbonate. Appl Rad Isot. 1995:46:887-891.
[8] Gad S C, Conroy W J, McKelvey J A, Turney K A. Behavioral and Neuropuarmacological Toxicology of the Macrocyclic Ether 18-Ckown-6. Drug and chemical toxicology. 1978 Jan. 1; 1(4):339-53.
[9] Kuntzsch M, Lamparter D, Brüggener N, Müller M, Kienzle, G J, Reischl G. Development and successful validation of simple and fast TLC spot tests for determination of Kryptofix® 2.2. 2 and tetrabutylammonium in ¹⁸F-labeled radiopharmaceuticals. Pharmaceuticals. 2014; 7:621-633.
[10] Scott P J, Kilbourn M R. Determination of residual Kryptofix 2.2. 2 levels in [¹⁸F]-labeled radiopharmaceuticals for human use. Applied Radiation and Isotopes. 2007 Dec. 1; 65(12):1359-62.
[11] Ferrieri R A, Schlyer D J, Alexoff D L, Fowler J S, Wolf A P. Direct analysis of Kryptofix 2.2. 2 in 18FDG by gas chromatography using a nitrogen-selective detector. Nucl med biol. 1993; 20:367-369.
[12] Nakao R, Ito T, Yamaguchi M, Suzuki K. Simultaneous analysis of FDG, CIDG, and Kryptofix 2.2.2 in [¹⁸F]FDG preparation by high-performance liquid chromatography with UV detection. Nucl med biol. 2008; 35:239-244.
[13] Tarn M D, Esfahani M M, Patinglag L, Chan Y C, Buch J X, Onyije C C, Gawne P J, Gambin D J, Brown N J, Archibald S J, Pamme N. Microanalytical devices towards integrated quality control testing of [¹⁸F]FDG radiotracer. In Proceedings of the 21st International Conference on Miniaturized Systems for Chemistry and Life Sciences, Savannah, Ga., USA 2017 October (pp. 22-26).
[14] He M Y, Sun L Q, Luo A Q. Spectrophotometric Determination of Kryptofix 2.2.2 Using TCNQ. Adv Materials Res. 2012; 581:1156-1159.
[15] Rodnick M E, Brooks A F, Hockley B G, Henderson B D, Scott P J. A fully-automated one-pot synthesis of [¹⁸F] fluoromethylcholine with reduced dimethylaminoethanol contamination via [¹⁸F] fluoromethyl tosylate. Appl. Radiat. and Isot. 2013; 78:26-32.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method of detecting the presence or concentration of a phase transfer catalyst (PTC) in a sample, the method comprising:
(a) mixing a sample containing or suspected of containing a PTC with an aqueous solution comprising a potassium and/or sodium salt to provide an aqueous mixture;
(b) adding an organic solvent to the aqueous mixture to provide a biphasic mixture comprising an aqueous phase and an organic phase;
(c) mixing the biphasic mixture for a period of time;
(d) separating the organic phase from the aqueous phase; and
(e) analyzing the organic phase, thereby determining the presence or concentration of the PTC.

2. The method of claim 1, wherein the sample containing or suspected of containing a PTC comprises a radiopharmaceutical.

3. The method of claim 2, wherein the radiopharmaceutical comprises fluorine-18 (¹⁸F).

4. The method of claim 3, wherein the radiopharmaceutical is selected from the group consisting of [¹⁸F]2-fluoro-2-deoxy-D-glucose (FDG) [¹⁸F]sodium fluoride; [¹⁸F]3′-deoxy-3′-fluorothymidine (FLT), [¹⁸F]fluoromisonidazole, [¹⁸F]florbetaben, [¹⁸F]florbetapir, [¹⁸F]fluoro-ethyl-tyrosine (FET), [¹⁸F]flutemetamol, [¹⁸F]-fluorocholine (FCH), [¹⁸F]fluoroethylcholine (FECH), [¹⁸F]fallypride, and [¹⁸F]6-fluor-L-2,3-dihydroxyphenylalanine (FDOPA).

5. The method of claim 1, wherein the PTC is selected from a quaternary ammonium salt, a cryptand, and a crown ether.

6. The method of claim 5, wherein the PTC is selected from the group consisting of K-222, tetrabutylammonium hydrogen carbonate, and 18-crown-6.

7. The method of claim 1, wherein the organic solvent is dichloromethane.

8. The method of claim 1, wherein step (c) comprises vortexing the biphasic mixture for about 30 seconds.

9. The method of claim 1, wherein the potassium and/or sodium salt is the potassium or sodium salt of a chromophoric anion.

10. The method of claim 9, wherein the potassium and/or sodium salt is selected from the group consisting of potassium permanganate and sodium resazurin.

11. The method of claim 1, wherein step (e) comprises measuring the light absorbance of the organic phase at one or more wavelengths of interest and comparing the light absorbance of the organic phase to the light absorbance of one or more standard solutions, wherein each of the one or more standard solutions comprises a known concentration of a complex of the PTC and the salt dissolved in the organic solvent.

12. The method of claim 11, wherein the potassium and/or sodium salt is selected from the group consisting of potassium permanganate and sodium resazurin, and the one or more wavelengths of interest is 532 nanometers (nm).

13. The method of claim 12, wherein the aqueous solution comprising the potassium and/or sodium salt comprises about 0.1 and about 0.5 molar (M) potassium permanganate, optionally wherein the aqueous solution comprising the potassium and/or sodium salt comprises about 0.2 M potassium permanganate.

14. The method of claim 12, wherein the measuring is performed using a spectrophotometric device comprising a green laser and a light detector.

15. The method of claim 14, wherein the device further comprises one or more of a reservoir for the organic phase, a microprocessor, a solid body for holding a sample reservoir in the path of a beam of light from the green laser, and a display for displaying one or more absorbance measurement values.

16. The method of claim 1, wherein step (e) comprises measuring the electrical conductivity of the organic phase and comparing the electrical conductivity of the organic phase to the electrical conductivity of one or more standard solutions, wherein each of the one or more standard solutions comprises a known concentration of a complex of the PTC and the salt dissolved in the organic solvent.

17. The method of claim 16, wherein the potassium and/or sodium salt comprises a mixture of sodium resazurin and potassium carbonate.

18. The method of claim 17, wherein the aqueous solution comprising the potassium or sodium salt comprises between about 0.02 and about 0.06 molar (M) sodium resazurin and between about 0.02 and about 0.06 M potassium carbonate.

19. The method of claim 18, wherein the aqueous solution comprises equimolar concentrations of the sodium resazurin and the potassium carbonate, optionally wherein both the sodium resazurin and the potassium carbonate have a concentration of about 0.05 M.

20. The method of claim 16, wherein the measuring is performed with a photodiode resistor or a multimeter.

21. The method of claim 1, wherein the sample has a volume of between about 50 microliters (μL) and about 100 μL, and the aqueous solution comprising a potassium and/or sodium salt has a volume of about 50 μL.

22. The method of claim 20, wherein adding an organic solvent comprises adding about 1 milliliter (mL) of the organic solvent.

23. The method of claim 1, wherein the potassium and/or sodium salt is the potassium or sodium salt of a chromophoric anion and wherein step (e) comprises visually comparing the color of the organic phase to the color of one or more standard solutions, wherein each of the one or more standard solutions comprises a known concentration of a complex of the PTC and the salt dissolved in the organic solvent.

24. A method of conducting a quality control test on a radiopharmaceutical, wherein the method comprises:
(a) mixing an aliquot of a radiopharmaceutical solution with an aqueous solution comprising a potassium and/or sodium salt to provide an aqueous mixture;
(b) adding an organic solvent to the aqueous mixture to provide a biphasic mixture comprising an aqueous phase and an organic phase;
(c) mixing the biphasic mixture for a period of time;
(d) separating the organic phase from the aqueous phase; and
(e) analyzing the organic phase, thereby determining the concentration of a residual phase transfer catalyst (PTC) in the radiopharmaceutical.

25. The method of claim 24, wherein the analyzing comprises (i) measuring an optical absorbance of the organic phase or an electrical conductivity of the organic phase, and (ii) comparing the optical absorbance or electrical conductivity to an optical absorbance or electrical conductivity of one or more standard solutions, wherein each of the one or more standard solutions comprises a known concentration of the PTC complexed to the potassium and/or sodium salt.

26. The method of claim 24, wherein the residual PTC is selected from K-222, tetrabutylammonium hydrogen carbonate, and 18-crown-6.

27. The method of claim 24, wherein the radiopharmaceutical is selected from the group consisting of [¹⁸F]2-fluoro-2-deoxy-D-glucose (FDG) [¹⁸F]sodium fluoride; [¹⁸F]3′-deoxy-3′-fluorothymidine (FLT), [¹⁸F]fluoromisonidazole, [¹⁸F]florbetaben, [¹⁸F]florbetapir, [¹⁸F]fluoro-ethyl-tyrosine (FET), [¹⁸F]flutemetamol, [¹⁸F]-fluorocholine (FCH), [¹⁸F]fluoroethylcholine (FECH), [¹⁸F]fallypride, and [¹⁸F]6-fluor-L-2,3-dihydroxyphenylalanine (FDOPA).
